(19) United States
(12) Patent Application Publication
(10) Pub. No.: US 2008/0063672 A1
(43) Pub. Date: Mar. 13, 2008

(54) Device for applying a cosmetic product

(76) Inventors: Antonio Mandelli, Cernusco Sul Naviglio (IT); Andrea Ferrari, Monza (IT)

Correspondence Address: BLANK ROME LLP, 600 NEW HAMPSHIRE AVENUE, N.W., WASHINGTON, DC, 20037, US

(21) Appl. No.: 11/529,581

(22) Filed: Sep. 29, 2006

(30) Foreign Application Priority Data
Sep. 8, 2006 (IT) MI2006A001712

(51) Int. Cl. B65D 35/28; A61K 8/02

(52) U.S. Cl. 424/401; 222/101

(57) ABSTRACT

A device for applying a cosmetic product, such as a make-up product (blush or “fard”, face powder, corrector, base make-up, eye-shadow, etc.) is defined by a cap housing a solid mass of cosmetic product having a contact surface, and by a brush housed removably inside the cap and movable inside the cap to cooperate with the contact surface; the cosmetic product is defined by a particle aggregate having a composition including a lipid phase and a powder phase, and the lipid phase ranges in quantity between no less than approximately 10% and no more than approximately 80% by weight.

The present invention relates to a device for applying a cosmetic product, and is particularly, though not exclusively, suitable for applying make-up products such as blush or “fard”, face powder, corrector, base make-up, eye-shadow, etc.

### BACKGROUND OF THE INVENTION

As is known, blush or “fard” is normally sold in compacted powder form in a container, and is brushed onto the face. To avoid carrying both the brush and the container (and possibly various containers of different products of different colors and shades), especially outside the home or when travelling, applicators are known in which the brush is housed inside a cap with the blush fitted on the bottom; the brush is moved inside the cap to remove and deposit a certain amount of powder on the brush; and the brush is then removed from the cap to apply the make-up.

Known blush compositions, which are substantially powdery, mainly comprising a powder excipient, such as talc, and colorants, have proved unsuitable for use in portable applicators of the type described above, which in fact call for a cosmetic product which, though substantially powdery, can be compacted, and remains firmly compacted while at the same time being effectively removable in suitable quantities by the abrasive action of the bristles on the brush. The same also applies to other cosmetic products, which are unsuitable for use with portable applicators of the type described.

### SUMMARY OF THE INVENTION

It is an object of the present invention to provide a device for applying a cosmetic product, in particular blush or “fard” or other make-up products, designed to eliminate the drawbacks of the known state of the art. In particular, it is an object of the invention to provide an applicator device which is not only portable, compact, and easy to use, but also fully effective.

According to the present invention, there is provided a device for applying a cosmetic product, as claimed in the attached Claims.

The device according to the invention is extremely compact, easy to use, and, at the same time, highly effective, by virtue of the optimum powdery nature and almost pasty consistency of the cosmetic product, which may therefore be compacted and housed inside the cap, remains firmly compacted to prevent loss prior to use, and, at the same time, can be effectively removed in suitable quantities simply by the abrasive action of the bristles on the brush.

### BRIEF DESCRIPTION OF THE DRAWINGS

A number of non-limiting embodiments of the invention will be described by way of example with reference to the accompanying drawings, in which:

FIG. 1 shows a schematic, partly sectioned, exploded view of an applicator device in accordance with the invention;

FIG. 2 shows a variation of the FIG. 1 device.

### DETAILED DESCRIPTION OF THE INVENTION

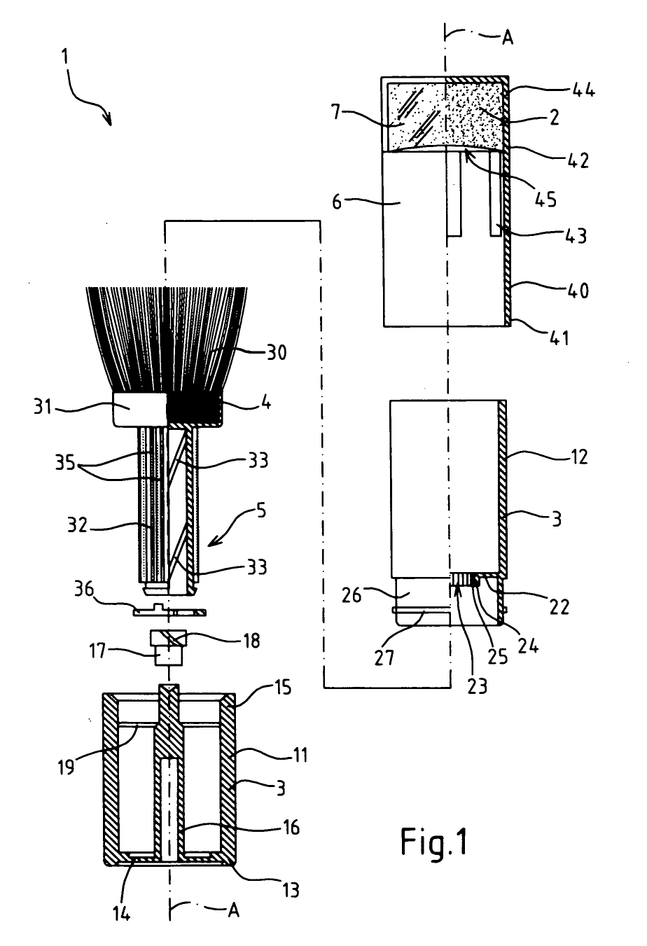

Number 1 in FIG. 1 indicates as a whole a device for applying a cosmetic product 2, such as blush (also called “rouge” or “fard”).

Device 1 comprises a casing 3 extending along an axis A; a brush 4 housed inside casing 3 and movable along axis A; a mechanism 5 for moving brush 4; and a cap 6 fitted removably to casing 3 and having at least one cartridge 7 containing a quantity of cosmetic product 2 (blush).

Casing 3 comprises two tubular, substantially cylindrical members 11, 12 fitted in rotary manner to each other. Member 11 has one axial end 13 closed by a bottom wall 14, and one open axial end 15, and houses a rod 16 fixed integrally to bottom wall 14 and projecting from bottom wall 14 along axis A; a free end of rod 16 is fitted integrally with a cylindrical collar 17 having, on the outside, two curved, diametrically opposite grooves 18 defining respective thread segments; and, close to end 15, member 11 has an inner circumferential annular groove 19. Member 12 extends between two open axial ends, and houses a radially inner flange 22 having a central through hole 23 bounded by a saw-toothed inner peripheral edge 24 with a number of radially inner teeth 25; and member 12 comprises an end portion 26 inserted inside open end 15 of member 11, and having an annular projection 27 which engages groove 19 to form a rotary connection between the two members 11, 12.

Brush 4 comprises a number of bristles 30 fixed to a supporting ring 31; and ring 31 is located at one end of a tubular bar 32 housed inside casing 3 and fitted through hole 23.

Mechanism 5 is defined by a screw coupling between bar 32 and collar 17. Bar 32 is hollow, is fitted onto rod 16, and has, inside, two diametrically opposite, helical projections 33 defining respective threads which engage grooves 18; an outer lateral surface of bar 32 has a number of longitudinal grooves 35 engaged by respective teeth 25 on saw-toothed edge 24; bar 32 and member 12 are connected angularly, while bar 32 slides along axis A with respect to member 12 and hence with respect to casing 3. At the end opposite the end fitted with ring 31 supporting bristles 30, bar 32 is fitted with a radially outer stop ring 36.

Cap 6 is defined by a tubular, substantially cylindrical body 40 having an open axial end 41 for connection to casing 3, and an axial end 42 having cartridge 7. Cap 6 is fitted removably to member 12, and has retaining projections 43 inside, which cooperate with the outer surface of member 12 to retain cap 6 on casing 3. In the FIG. 1 example, cartridge 7 is defined by a transparent-walled end portion 44 of body 40, and is formed in one piece with body 40.

Cartridge 7 contains a quantity of cosmetic product 2 having a contact surface 45 facing end 41. Cosmetic product 2 is defined by a solid particle aggregate having a composition comprising a lipid phase and a powder phase, and in which the lipid phase ranges in quantity between no less than approximately 10% and no more than approximately 80% by weight. Cosmetic product 2 preferably comprises approximately 20 to 70% by weight, and more preferably approximately 30 to 60% by weight of lipid phase.

The lipid phase substantially comprises known components, such as natural and/or synthetic esters, polyolefins, polyethylene wax, microcrystalline wax, etc., which act as emollients or softeners, skin conditioning agents, film formers (filmogens), viscosity control agents, etc.

The powder phase includes one or more powder excipients, in particular, for example, talc, silica, mica or other inorganic powders; one or more colorants (e.g. metal oxides, such as titanium oxide, ferrous oxide, etc.); at least one binder, e.g. a polymer binder; and possibly known cosmetic additives, such as sun filters, preservatives, antioxidants, emulsifiers, etc.

Cosmetic product 2 is substantially a solid suspension of the powder phase—bound with at least one (e.g. polymer) binder—in the lipid phase, and has an almost pasty or creamy consistency.

More specifically, cosmetic product 2 includes talc bound with an olefin-acrylic copolymer, preferably an alkene/methacrylate copolymer, and more preferably an ethylene/methacrylate copolymer.

Cosmetic product 2 also includes a metal stearate, in particular a titanium stearate, preferably titanium triisostearate, and more preferably isopropyl titanium triisostearate.

In a preferred embodiment, the talc, polymer binder (ethylene/methacrylate copolymer) and metal stearate (isopropyl titanium triisostearate) are premixed to form an association which is then added to the other components of cosmetic product 2.

Table 1 shows the main components of non-limiting examples of compositions of cosmetic product 2; and non-limiting examples of complete compositions are shown in Table 2.

TABLE 1

| | QUANTITY | |
|---|---|---|
| COMPONENT | (% by weight) | EXAMPLES |
| LIPID PHASE | 10 ÷ 80 | |
| EMOLLIENTS (SOFTENERS) | 20 ÷ 40 | CETEARYL ETHYLHEXANOATE; |
| | | ISOSTEARYL NEOPENTANOATE |
| SKIN CONDITIONING | 15 ÷ 35 | HYDROGENATED POLYDECENE; |
| AGENTS | | COPERNICIA CERIFERA |
| | | (CARNAUBA) WAX; |
| | | PENTAERYTHRITYL |
| | | STEARATE/CAPRATE/CAPRYLATE |
| | | ADIPATE |
| OTHER AGENTS | 3 ÷ 15 | filmogens (e.g. |
| | | POLYETHYLENE); viscosity |
| | | control agents (e.g. |
| | | MICROCRYSTALLINE WAX) |
| POWDER PHASE | 20 ÷ 90 | |
| POWDER EXCIPIENT | 5 ÷ 15 | TALC |
| POLYMER BINDER | 1 ÷ 3 | ETHYLENE/METHACRYLATE |
| | | COPOLYMER |
| SURFACE PROPERTY | 0.1 ÷ 1 | ISOPROPYL TITANIUM |
| CONTROL AGENT | | TRIISOSTEARATE |
| COLORANTS | 1 ÷ 20 | TITANIUM DIOXIDE; FERROUS |
| | | OXIDE; MICA |
| OTHER ADDITIVES | 0 ÷ 5 | sunscreen agents; |
| | | preservatives; |
| | | antioxidants; emulsifiers; |
| | | etc. |

TABLE 2

| | QUANTITY | |
|---|---|---|
| COMPONENT | (% by weight) | FUNCTION |
| CETEARYL ETHYLHEXANOATE | 20 ÷ 35 | emollient |
| HYDROGENATED POLYDECENE | 10 ÷ 20 | skin conditioning agent |
| ISOSTEARYL NEOPENTANOATE | 4 ÷ 12 | emollient |
| COPERNICIA CERIFERA | 4 ÷ 12 | skin conditioning agent |
| (CARNAUBA) WAX | | |
| POLYETHYLENE | 3 ÷ 10 | film former (filmogen) |
| PENTAERYTHRITYL | 2 ÷ 6 | skin conditioning agent |
| STEARATE/CAPRATE/CAPRYLATE | | |
| ADIPATE | | |
| MICROCRYSTALLINE WAX | 1 ÷ 5 | viscosity control agent |
| TALC | 5 ÷ 15 | powder |
| ETHYLENE/METHACRYLATE | 1 ÷ 3 | binder |
| COPOLYMER | | |
| ISOPROPYL TITANIUM | 0.1 ÷ 1 | surface property |
| TRIISOSTEARATE | | control agent |
| TITANIUM DIOXYDE | 4 ÷ 8 | colorant |
| IRON OXIDES | 4 ÷ 8 | colorant |
| MICA | 1 ÷ 5 | colorant |
| ETHYLHEXYL METHOXYCINNAMATE | 0 ÷ 1 | sunscreen agent |
| METHYLPARABEN | 0 ÷ 1 | preservative |
| PROPYLPARABEN | 0 ÷ 1 | preservative |
| ASCORBYL PALMITATE | 0 ÷ 0.1 | antioxidant |
| LECITHIN | 0 ÷ 0.1 | emulsifying agent |
| TOCOPHEROL | 0 ÷ 0.1 | antioxidant |
| CITRIC ACID | 0 ÷ 0.1 | antioxidant |

In actual use, with cap 6 fitted to casing 3, the user rotates member 11 so that brush 4 cooperates with contact surface 45 of cosmetic product 2; with cap 6 in place and by rotating member 11 alternately clockwise/anticlockwise, brush 4 rubs against contact surface 45 and removes cosmetic product 2 which is deposited on brush 4. The user then removes cap 6 and, by rotating member 11 with respect to member 12, extracts brush 4 to apply cosmetic product 2 to the face.

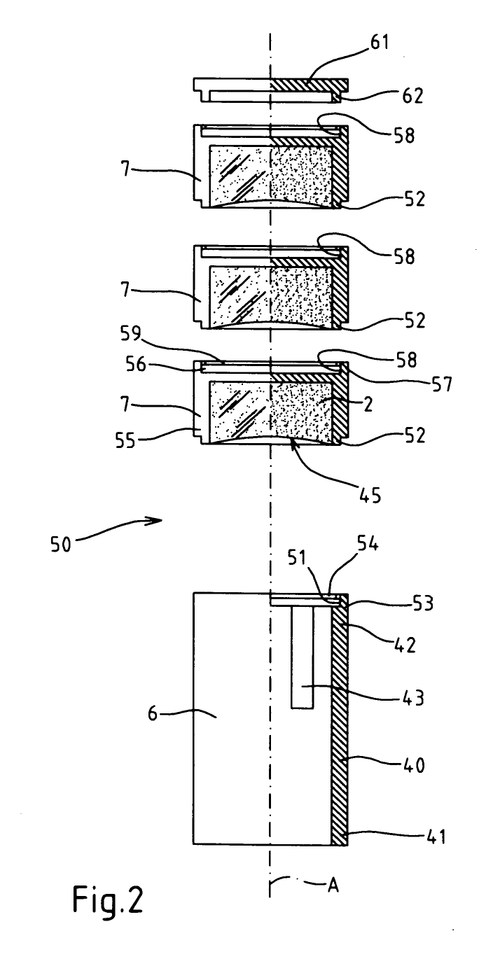

FIG. 2 shows a modified cap 6 (the rest of device 1 is the same as described above). In this variation, cosmetic product 2 is contained in a cartridge 7 which, as opposed to being formed in one piece with body 40 of cap 6, is connected removably to cap 6, by releasable, e.g. male/female, connecting members 50, so that it can be replaced with another cartridge 7.

Both ends 41, 42 of cap 6 are open, and members 50 comprise an insertion seat 51 defined by end 42 of cap 6; and a connecting edge 52, on cartridge 7, which is force-fitted or clicks inside seat 51. Seat 51 is bounded by a substantially cylindrical collar 53, possibly having an annular, radially inner projection 54; connecting edge 52 is formed radially inwards on an axial end 55 of cartridge 7, at contact surface 45 of cosmetic product 2; and cartridge 7 is defined by a hollow, substantially cup-shaped body preferably made of transparent plastic material.

An axial end 56 of cartridge 7, opposite end 55, has a radially outer edge 57, which defines an insertion seat 58 similar to seat 51, and may have an annular, radially inner projection 59. Cartridge 7 therefore has an insertion seat 58 and a connecting edge 52 at respective opposite axial ends, to permit stacking of a number of cartridges 7. A cover 61, with a connecting edge 62 insertable inside a seat 51 or 58, is provided to complete the stack of cartridges 7.

Clearly, changes may be made to the applicator device and cosmetic product compositions as described and illustrated herein without, however, departing from the scope of the accompanying Claims.

In particular, though reference is made purely by way of example to blush compositions, the device according to the invention may obviously be used to apply other cosmetic products; the compositions indicated in fact may be modified, while still maintaining the general characteristics indicated above, to form different cosmetic products, such as face powder, corrector, powdered base make-up, eye-shadow, etc.

1) A device (1) for applying a cosmetic product (2), comprising a cap (6) housing a solid mass of cosmetic product (2) having a contact surface (45); and a brush (4) housed removably inside the cap and movable inside the cap to cooperate with the contact surface; the device being characterized in that the cosmetic product (2) is defined by a particle aggregate having a composition comprising a lipid phase and a powder phase, and the lipid phase ranges in quantity between approximately 10 and 80% by weight.

2) A device as claimed in claim 1, characterized in that the cosmetic product comprises approximately 20 to 70% by weight of lipid phase.

3) A device as claimed in claim 1, characterized in that the cosmetic product comprises approximately 30 to 60% by weight of lipid phase.

4) A device as claimed in claim 1, characterized in that the powder phase includes one or more powdered excipients; one or more colorants; and at least one binder, in particular a polymer binder.

5) A device as claimed in claim 1, characterized in that the cosmetic product is substantially a solid suspension of the powder phase—bound with at least one binder, e.g. a polymer binder—in the lipid phase.

6) A device as claimed in claim 1, characterized in that the cosmetic product includes talc bound with an olefin-acrylic copolymer.

7) A device as claimed in claim 6, characterized in that the talc is bound with an alkene/methacrylate copolymer.

8) A device as claimed in claim 7, characterized in that the talc is bound with an ethylene/methacrylate copolymer.

9) A device as claimed in claim 1, characterized in that the cosmetic product includes a metal stearate.

10) A device as claimed in claim 9, characterized in that the cosmetic product includes a titanium stearate.

11) A device as claimed in claim 10, characterized in that the cosmetic product includes titanium triisostearate, in particular isopropyl titanium triisostearate.

12) A device as claimed in claim 1, characterized in that the cosmetic product includes an association of talc, ethylene/methacrylate copolymer, and titanium triisostearate, in particular isopropyl titanium triisostearate.

13) A device as claimed in claim 1, characterized in that the powder phase includes an association of talc and at least one binder, in particular a polymer binder; the association being premixed and then added to the lipid phase.

14) A device as claimed in claim 13, characterized in that the association of talc and at least one binder, in particular a polymer binder, also includes a titanium stearate, in particular isopropyl titanium stearate.

15) A device as claimed in claim 1, characterized in that the cosmetic product is a make-up product, in particular blush or “fard”, face powder, corrector, powdered base make-up, eye-shadow.

16) A device as claimed in claim 1, characterized in that the brush (4) is housed in a casing (3), and the cap (6) is fitted removably to the casing; the device comprising a mechanism (5) for moving the brush (4) along an axis (A) of the casing (3), so that the brush cooperates with the contact surface (45) of the cosmetic product (2).

17) A device as claimed in claim 1, characterized in that the cosmetic product (2) is contained in a cartridge (7) fitted removably to the cap (6) by releasable connecting members (50) so as to be replaceable with another cartridge (7).

18) A device as claimed in claim 1, characterized by comprising one or more removable cartridges (7) replaceable with one another; and connecting members (50) for securing the cartridges (7) releasably to one another and to the cap (6).
